(19) United States
(12) Patent Application Publication
(10) Pub. No.: US 2006/0264929 A1
(43) Pub. Date: Nov. 23, 2006

(54) Surgical system

(75) Inventors: Colin C.O. Goble, Surrey (GB); Kester J. Batchelor, Gwent (GB); Mark G. Marshall, Berkshire (GB)

Correspondence Address: NIXON & VANDERHYE, PC, 901 NORTH GLEBE ROAD, 11TH FLOOR, ARLINGTON, VA, 22203, US

(73) Assignee: Gyrus Group PLC, Berkshire (GB)

(21) Appl. No.: 11/403,939

(22) Filed: Apr. 14, 2006

Related U.S. Application Data
(63) Continuation-in-part of application No. 11/028,573, filed on Jan. 5, 2005.
(63) Continuation-in-part of application No. 11/210,671, filed on Aug. 25, 2005.
Application No. 11/210,671 is a division of application No. 10/324,069, filed on Dec. 20, 2002, now Pat. No. 6,942,662.
Application No. 10/324,069 is a continuation-in-part of application No. 10/105,811, filed on Mar. 21, 2002, now Pat. No. 6,832,998.

(30) Foreign Application Priority Data
Dec. 27, 2001 (GB) 0130975.6
Jul. 3, 2002 (GB) 0215402.9
Mar. 15, 2002 (GB) 0206207.3
Nov. 24, 2004 (GB) 0425842.2

(51) Int. Cl. A61B 18/14

(52) U.S. Cl. 606/48; 606/50

(57) ABSTRACT

An electrosurgical instrument includes a hand piece, an electrode assembly comprising one or more electrodes attached to the hand piece, and connection means for connecting the hand piece to an electrosurgical generator. The hand piece comprises a housing, fluid supply lines for directing a cooling fluid to and from the electrode assembly, and a pump for driving cooling fluid through the fluid supply lines. An electrosurgical cutting blade comprises a first electrode, a second electrode, and an electrical insulator separating the first and second electrodes. The first and second electrode have dissimilar characteristics, such that the first electrode is encouraged to become an active electrode and the second electrode is encouraged to become a return electrode. In use, a thermal differential is established between the first and second electrodes, either by thermally insulating the second electrode from the first electrode, and/or by transferring heat away from the second electrode.

### RELATED APPLICATIONS

This application is a continuation-in-part of application Ser. No. 11/028,573, filed Jan. 5, 2005, and of application Ser. No. 11/210,671, filed Aug. 25, 2005, which is a divisional of application Ser. No. 10/324,069, filed Dec. 20, 2002, now U.S. Pat. No. 6,942,662 B2, which is a continuation-in-part of application Ser. No. 10/105,811, filed Mar. 21, 2002, now U.S. Pat. No. 6,832,998 B2, the entire contents of which are hereby incorporated by reference in this application.

### BACKGROUND OF THE INVENTION

1. Field of the Invention

The present invention relates to an electrosurgical surgical instrument comprising a handpiece including one or more electrosurgical electrodes. The present invention also relates to a bipolar electrosurgical cutting device such as a scalpel blade, and to an electrosurgical system comprising an electrosurgical generator and a bipolar electrosurgical cutting device. Such instruments and systems are commonly used for the cutting and/or coagulation of tissue in surgical intervention, most commonly in “keyhole” or minimally-invasive surgery, but also in “open” or “laparoscopic assisted” surgery.

It is known to provide an electrosurgical instrument with a cooling system for preventing excess temperatures being developed at the electrode or electrodes. These fall into two categories. The first category includes instruments with a circulating cooling fluid. Examples are U.S. Pat. No. 3,991,764, U.S. Pat. No. 4,202,336, U.S. Pat. No. 5,647,871 and EP 0246350A. It should be noted that, with each of these systems, some or all of the fluid reservoir, pump and fluid supply lines are located externally of the electrosurgical handpiece. The second category includes instruments with heat pipes. Examples are U.S. Pat. No. 6,733,501, U.S. Pat. No. 6,544,264, U.S. Pat. No. 6,503,248, U.S. Pat. No. 6,206,876, and U.S. Pat. No. 6,074,389.

2. Description of Related Art

Electrosurgical cutting devices generally fall into two categories, monopolar and bipolar. In a monopolar device a radio frequency (RF) signal is supplied to an active electrode which is used to cut tissue at the target site, an electrical circuit being completed by a grounding pad which is generally a large area pad attached to the patient at a location remote from the target site. In contrast, in a bipolar arrangement both an active and a return electrode are present on the cutting device, and the current flows from the active electrode to the return electrode, often by way of an arc formed therebetween.

An early example of a bipolar RF cutting device is U.S. Pat. No. 4,706,667 issued to Roos, in which the return or “neutral” electrode is set back from the active electrode. Details for the areas of the cutting and neutral electrodes are given, and the neutral electrode is said to be perpendicularly spaced from the active electrode by between 5 and 15 mm. In a series of patents including U.S. Pat. No. 3,970,088, U.S. Pat. No. 3,987,795 and U.S. Pat. No. 4,043,342, Morrison describes a cutting/coagulation device which has “sesquipolar” electrode structures. These devices are said to be a cross between monopolar and bipolar devices, with return electrodes which are carried on the cutting instrument, but which are preferably between 3 and 50 times larger in area than the cutting electrode. In one example (U.S. Pat. No. 3,970,088) the active electrode is covered with a porous, electrically-insulating layer, separating the active electrode from the tissue to be treated and causing arcing between the electrode and the tissue. The insulating layer is said to be between 0.125 and 0.25 mm (0.005 and 0.01 inches) in thickness.

In another series of patents (including U.S. Pat. No. 4,674,498, U.S. Pat. No. 4,850,353, U.S. Pat. No. 4,862,890 and U.S. Pat. No. 4,958,539) Stasz proposed a variety of cutting blade designs. These were designed with relatively small gaps between two electrodes such that arcing would occur therebetween when an RF signal was applied to the blade, the arcing causing the cutting of the tissue. Because arcing was designed to occur between the electrodes, the typical thickness for the insulating material separating the electrodes was between 0.025 and 0.075 mm (0.001 and 0.003 inches).

### BRIEF SUMMARY OF THE INVENTION

In one aspect, it is an aim of the present invention to provide an improvement over prior art electrosurgical instruments with cooling systems.

Accordingly, there is provided an electrosurgical instrument comprising a handpiece, an electrode assembly comprising one or more electrodes attached to the handpiece, and connection means for connecting the handpiece to an electrosurgical generator, the handpiece comprising a housing, fluid supply lines for directing a cooling fluid to and from the electrode assembly, and a pump for driving cooling fluid through the fluid supply lines, the pump and the fluid supply lines both being wholly contained within the housing.

The above arrangement provides the advantages of a circulating cooling fluid system, without the requirement for additional coolant lines and equipment external to the instrument handpiece. The handpiece can be supplied together with a reservoir of cooling fluid, or alternatively this can be assembled within the handpiece immediately prior to the instrument being used. In a preferred arrangement the housing also contains a reservoir of cooling fluid, and there are two possible arrangements for the fluid reservoir, a first arrangement in which the reservoir is not connected to the fluid supply lines, and a second arrangement in which the reservoir is connected to the fluid supply lines. In this way, the instrument can be supplied with all of the necessary components, and yet the reservoir need not be connected to the supply lines until the instrument is ready for use. This minimizes the risk of contamination of the cooling fluid or the corrosion of other components by the fluid, thereby increasing the acceptable shelf-life of the instrument.

In one convenient arrangement, the housing is such that the reservoir is movable between first and second positions, the first position being in which the reservoir is not connected to the fluid supply lines, and the second position being in which the reservoir is connected to the fluid supply lines. In this way, the fluid reservoir can be moved into position, e.g. by a sliding movement, either when the instrument is manufactured, or alternatively immediately prior to the first use of the instrument.

Conveniently, the electrode assembly comprises at least two electrodes separated by an insulating spacer. The electrode assembly preferably comprises three electrodes provided in a sandwich structure with insulating layers therebetween. In one convenient arrangement the electrode assembly is in the form of a relatively flat blade, as described in our published patent application EP 1458300.

The pump is preferably driven by an electric motor, typically a synchronous motor. In one convenient arrangement, the electric motor constitutes the pump. The motor conveniently includes a spindle on which is provided a paddle, the paddle being rotatable by the motor. The rotation of the paddle causes the cooling fluid to be driven though the fluid supply lines. Other types of pump, including those known for use with electronic equipment such as computers, may be suitable for use with this electrosurgical instrument. One such pump is an electrokinetic pump sold under the trade name “Cooligy” by Cooligy Inc. of Mountain View, Calif.

Conceivably, the pump may require priming before it is used for the first time. Mechanical or other types of pump priming mechanisms are well known in the art. If a pump priming mechanism is provided, this may extend from the housing without departing from the scope of the present invention.

The invention further provides an electrosurgical system comprising an electrode assembly comprising one or more electrodes, a handpiece to which the electrode assembly is secured, an electrosurgical generator for supplying a radio frequency voltage signal to the electrode assembly, and a cooling system for cooling the electrode assembly, the cooling system including fluid supply lines and a pump for driving cooling fluid through the fluid supply lines, the cooling system being wholly contained within the handpiece and the electrode assembly.

The invention also provides an electrosurgical handpiece comprising a housing, first connection means for attaching an electrode assembly to the handpiece, and second connection means for connecting the handpiece to an electrosurgical generator, the handpiece also including fluid supply lines for directing a cooling fluid to and from the electrode assembly, and a pump for driving cooling fluid through the fluid supply lines, the pump and the fluid supply lines both being wholly contained within the housing of the handpiece.

In another aspect, the present invention seeks to provide a bipolar cutting blade which is an improvement over the prior art. Accordingly, there is provided an electrosurgical system comprising a bipolar cutting blade, a handpiece to which the cutting blade is secured, and an electrosurgical generator for supplying a radio frequency voltage signal to the cutting blade, the cutting blade comprising first and second electrodes, and an electrical insulator spacing apart the electrodes, the spacing being between 0.25 mm and 3.0 mm, and the electrosurgical generator being adapted to supply a radio frequency voltage signal to the cutting blade which has a substantially constant peak voltage value, the relationship between the peak voltage value and the spacing between the electrodes being such that the electric field intensity between the electrodes is between 0.1 volts/μm and 2.0 volts/μm, the first electrode having a characteristic which is dissimilar from that of the second electrode such that the first electrode is encouraged to become an active electrode and the second electrode is encouraged to become a return electrode.

By the term “blade”, there is herein meant to include all devices which are designed such that both the active cutting electrode and the return electrode are designed to enter the incision made by the instrument. It is not necessary that the cutting device is only capable of making an axial incision, and indeed it will be shown below that embodiments of the present invention are capable of removing tissue in a lateral direction.

The first important feature of the present invention is that the spacing between the electrodes and the electric field intensity therebetween is carefully controlled such that there is no direct arcing between the electrodes in the absence of tissue. For the purposes of this specification, the spacing between the electrodes is measured in terms of the shortest electrical path between the electrodes. Thus, even if electrodes are adjacent on to another such that the straight-line distance therebetween is less than 0.25 mm, if the insulator separating the electrodes is such that this straight line is not available as a conductive pathway, then the “spacing” between the electrodes is the shortest available conductive path between the electrodes. The electric field intensity between the electrodes is preferably between 0.15 volts/μm and 1.5 volts/μm, and typically between 0.2 volts/μm and 1.5 volts/μm. In one preferred arrangement, the spacing between the first and second electrodes is between 0.25 mm and 1.0 mm, and the electric field intensity between the electrodes is between 0.33 volts/μm and 1.1 volts/μm. Preferably, the electric field intensity is such that the peak voltage between the first and second electrodes is less than 750 volts. This ensures that the field intensity is sufficient for arcing to occur between the first electrode and the tissue, but not directly between the first and second electrodes.

However, even where direct arcing between the electrodes is prevented, there is still a potential problem if the two electrodes are similar in design. In a bipolar cutting device only one of the electrodes will assume a high potential to tissue (and become the “active” electrode), with the remaining electrode assuming virtually the same potential as the tissue (becoming the “return” electrode). Where the first and second electrodes are similar, which electrode becomes the active can be a matter of circumstance. If the device is activated before becoming in contact with tissue, the electrode first contacting tissue will usually become the return electrode, with the other electrode becoming the active electrode. This means that in some circumstances one electrode will be the active electrode, and at other times the other electrode will be the active electrode. Not only does this make the device difficult for the surgeon to control (as it will be uncertain as to exactly where the cutting action will occur), but as it is likely that any particular electrode will at some time have been active.

When an electrode is active, there is a build up of condensation products on the surface thereof. This is not a problem when the electrode continues to be the active electrode, but it does make the electrode unsuitable for use as a return electrode. Thus, in the instance where two similar electrodes are employed, it is likely that, as each will at some times become active and at other times the return, the build up of products on both electrodes will lead to a decrease in performance of the instrument. Therefore, the present invention provides that the first electrode has a characteristic which is dissimilar from that of the second electrode, in order to encourage one electrode to assume preferentially the role of the active electrode.

The characteristic of the first electrode which is dissimilar from that of the second electrode conveniently comprises the cross-sectional area of the electrode, the cross-sectional area of the first electrode being substantially smaller than that of the second electrode. This will help to ensure that the first electrode (being of a smaller cross-sectional area) will experience a relatively high initial impedance on contact with tissue, while the relatively larger area second electrode will experience a relatively lower initial impedance on contact with tissue. This arrangement will assist in encouraging the first electrode to become the active and the second electrode to become the return.

The characteristic of the first electrode which is dissimilar from that of the second electrode alternatively or additionally comprises the thermal conductivity of the electrode, the thermal conductivity of the first electrode being substantially lower than that of the second electrode. In addition to the initial impedance, the rate of rise of the impedance is a factor influencing which electrode will become active. The impedance will rise with desiccation of the tissue, and the rate of desiccation will be influenced by the temperature of the electrode. By selecting an electrode material with a relatively low thermal conductivity, the electrode temperature will rise quickly as little heat is conducted away from the part of the electrode at which energy is delivered. This will ensure a relatively fast desiccation rate, producing a correspondingly fast rise in impedance and ensuring that the first electrode remains the active electrode.

The characteristic of the first electrode which is dissimilar from that of the second electrode may further comprise the thermal capacity of the electrode, the thermal capacity of the first electrode being substantially lower than that of the second electrode. As before, a low thermal capacity helps to maintain the temperature of the first electrode at a relatively high level, ensuring that it remains the active electrode.

According to a further aspect of the invention, there is provided an electrosurgical system comprising a bipolar cutting blade, a handpiece to which the cutting blade is secured, and an electrosurgical generator for supplying a radio frequency voltage signal to the cutting blade, the cutting blade comprising first and second electrodes, and an electrical insulator spacing apart the electrodes, the spacing being between 0.25 mm and 1.0 mm, and the electrosurgical generator being adapted to supply a radio frequency voltage signal to the cutting blade which has a substantially constant peak voltage value, the peak voltage value being respectively between 250 volts and 600 volts, the first electrode having a characteristic which is dissimilar from that of the second electrode such that the first electrode is encouraged to become an active electrode and the second electrode is encouraged to become a return electrode.

Given a particular electrode separation, it is highly desirable that the generator delivers the same peak voltages despite varying load conditions. Heavy loading of the blade may otherwise make it stall (as load impedance approaches source impedance, the voltage may otherwise halve), while light loading may otherwise result in voltage overshoots and direct arcing between the electrodes.

The invention also resides in a bipolar cutting blade comprising first and second electrodes and an electrical insulator spacing apart the electrodes, the first electrode having a characteristic which is dissimilar from that of the second electrode such that the first electrode is encouraged to become an active electrode and the second electrode is encouraged to become a return electrode, the spacing between the electrodes being between 0.25 mm and 1.0 mm, such that when the electrodes are in contact with tissue and an electrosurgical cutting voltage is applied therebetween, arcing does not occur directly between the electrodes, there also being provided means for ensuring that the temperature of the second electrode does not rise above 70° C.

As well as ensuring that the second electrode does not become active, it is also important to ensure that the temperature of the second electrode does not rise above 70° C., the temperature at which tissue will start to stick to the electrode. The means for ensuring that the temperature of the second electrode does not rise above 70° C. conveniently comprises means for minimising the transfer of heat from the first electrode to the second electrode. One way of achieving this is to ensure that the first electrode is formed from a material having a relatively poor thermal conductivity, preferably less than 20 W/m.K. By making the first electrode a poor thermal conductor, heat is not transferred effectively away from the active site of the electrode and across to the second electrode, thereby helping to prevent the temperature of the second electrode from rising.

Alternatively or additionally, the heat can be inhibited from transferring from the first electrode to the second electrode by making the electrical insulator separating the electrodes from a material having a relatively poor thermal conductivity, preferably less than 40 W/m.K. Again, this helps to prevent heat generated at the first electrode from transferring to the second electrode.

Another way of inhibiting the transfer of heat is to attach the first electrode to the electrical insulator in a discontinuous manner. Preferably, the first electrode is attached to the electrical insulator at one or more point contact locations, and/or is perforated with a plurality of holes such as to reduce the percentage contact with the electrical insulator.

A preferred material for the first electrode is tantalum. When tantalum is used for the active electrode, it quickly becomes coated with a layer of oxide material. This tantalum oxide is a poor electrical conductor, helping to ensure that the first electrode maintains its high impedance with respect to the tissue, and remains the active electrode.

Another way of helping to ensure that the temperature of the second electrode does not rise above 70° C. is to maximise the transfer of heat away from the second electrode. Thus any heat reaching the second electrode from the first electrode is quickly transferred away before the temperature of the second electrode rises inordinately. One way of achieving this is to form the second electrode from a material having a relatively high thermal conductivity, preferably greater than 150 W/m.K.

The second electrode may conveniently be provided with additional cooling means to remove heat there from, such as a heat pipe attached to the second electrode, or a cooling fluid constrained to flow along a pathway in contact with the second electrode. Whichever method is employed, it is advisable for there to be a temperature differential, in use, between the first and second electrodes of at least 50° C., and preferably of between 100 and 200° C.

Preferably, there is additionally provided a third electrode adapted to coagulate tissue. This coagulation electrode is conveniently attached to the second electrode with a further electrical insulator therebetween. It is necessary to ensure that the temperature of the coagulation electrode does not rise to too high a level, and so if the coagulation electrode is attached to the second electrode (which is designed in accordance with the present teaching to be a good thermal conductor), it is preferable to arrange that heat is easily transferred across the further electrical insulator. This can be achieved by making the further insulator from a material having a relatively high thermal conductivity, or more typically, if the further insulator is not a good thermal conductor, by ensuring that the further insulator is relatively thin, typically no more than around 50 μm. In this way the transfer of heat across the further electrical insulator is greater than 5 mW/mm².K.

In one arrangement, the second and third electrodes are formed as conductive electrodes on an insulating substrate. Thus both the second and third electrodes act as return electrodes when the blade is used to cut tissue with the first electrode. When the blade is used to coagulate tissue, a coagulating RF signal is applied between the second and third electrodes.

According to a further aspect of the invention, there is provided a bipolar cutting blade comprising first and second electrodes and an electrical insulator spacing apart the electrodes, the first electrode having a characteristic which is dissimilar from that of the second electrode such that the first electrode is encouraged to become an active electrode and the second electrode is encouraged to become a return electrode, the spacing between the electrodes being between 0.25 mm and 1.0 mm, such that when the electrodes are in contact with tissue and an electrosurgical cutting voltage is applied therebetween, arcing does not occur directly between the electrodes, there being additionally provided a third electrode adapted to coagulate tissue, the third electrode being separated from the second electrode by an additional insulator.

The second and third electrodes are conveniently provided in a side-by-side arrangement with the additional insulator therebetween. Alternatively, the second and third electrodes are provided as layers in a sandwich structure with the additional insulator therebetween. In one convenient arrangement the first, second and third electrodes are each provided as layers in a sandwich structure with layers of insulator therebetween.

In one arrangement a first one of the second and third electrodes is provided with a cut-out portion, and the other one of the second or third electrodes is provided with a protruding portion. Preferably, the cut-out portion of the one electrode accommodates the protruding portion of the other electrode, typically such that the protruding portion is flush with the electrode surrounding the cut-out portion.

Alternatively, the first, second and third electrodes are provided as layers in a sandwich structure with the first electrode being in the middle, there being layers of insulator between each of the electrodes. In one arrangement, the second and third electrodes are substantially semi-circular in cross-section, and the first electrode protrudes slightly beyond the periphery of the second and third electrodes.

According to a final aspect of the invention, there is provided a method of cutting tissue at a target site comprising providing a bipolar cutting blade comprising first and second electrodes and an electrical insulator spacing apart the electrodes, the first electrode having a characteristic which is dissimilar from that of the second electrode such that the first electrode is encouraged to become an active electrode and the second electrode is encouraged to become a return electrode; bringing the blade into position with respect to the target site such that the second electrode is in contact with tissue at the target site and the first electrode is adjacent thereto; supplying an electrosurgical cutting voltage to the cutting blade, the electrosurgical voltage and the spacing between the first and second electrodes being such that arcing does not occur in air between the first and second electrodes, but that arcing does occur between the first electrode and the tissue at the target site, current flowing through the tissue to the second electrode; and preventing heat build up at the second electrode such that the temperature of the second electrode does not rise above 70° C. Preferably, the method is such that both the first and second electrodes come into contact with tissue at the target site substantially simultaneously.

### BRIEF DESCRIPTION OF THE DRAWINGS

The invention will now be further described, by way of example only, with reference to the accompanying drawings, in which,

FIG. 1 is a schematic diagram of an electrosurgical system including an electrosurgical instrument constructed in accordance with the present invention;

FIGS. 2 and 3 are views, shown partly in section, of a handpiece forming part of the electrosurgical instrument of FIG. 1;

FIGS. 4 and 5 are sectional views of an alternative embodiment of handpiece forming part of the electrosurgical instrument of FIG. 1;

FIG. 6 is an enlarged sectional view of part of the handpiece of FIGS. 4 and 5;

FIG. 7 is a perspective view of an electrode assembly forming part of the electrosurgical instrument of FIG. 1;

FIG. 8 is a perspective view, shown partly in section, of the electrode assembly of FIG. 7;

FIG. 9 is a schematic sectional plan view of the electrode assembly of FIG. 7;

FIGS. 10A to 10F are perspective views showing the electrode assembly of FIG. 7 is various stages of assembly;

FIG. 11 is a schematic cross-sectional view of an electrosurgical cutting blade constructed in accordance with the present invention;

FIG. 12 is a schematic diagram showing the lateral cutting action of the blade of FIG. 11;

FIGS. 13a to 13d are schematic cross-sectional views of alternative embodiments of electrosurgical cutting blades constructed in accordance with the invention;

FIGS. 14a and 14b are schematic diagrams of electrosurgical cutting blades constructed in accordance with the present invention, incorporating cooling means; and

FIGS. 15a and 15b, and FIGS. 16 to 20 are alternative electrosurgical cutting blades constructed in accordance with the present invention, incorporating an additional coagulation electrode.

### DETAILED DESCRIPTION OF THE INVENTION

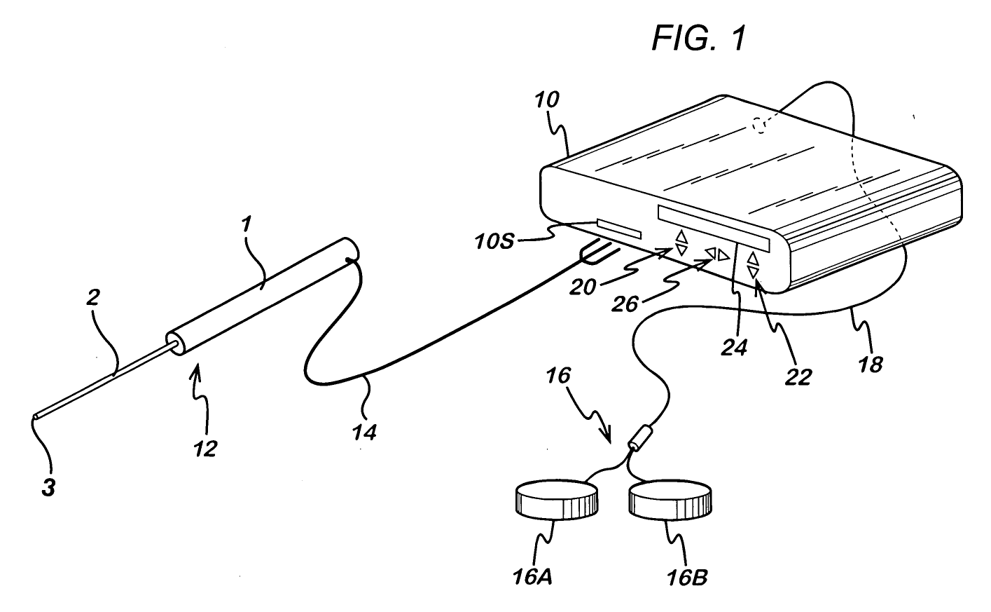

Referring to FIG. 1, a generator 10 has an output socket 10S providing a radio frequency (RF) output for an electrosurgical instrument 12 via a connection cord 14. Activation of the generator 10 may be performed from the instrument 12 via a connection cord 14, or by means of a footswitch unit 16, as shown, connected to the rear of the generator by a footswitch connection cord 18. In the illustrated embodiment, the footswitch unit 16 has two footswitches 16A and 16B for selecting a coagulation mode and a cutting mode of the generator 10 respectively. The front panel of the generator 10 has push buttons 20 and 22 for respectively setting parameters such as the coagulation and cutting power levels, which are indicated in a display 24. Push buttons 26 are provided as an alternative means for selection between coagulation and cutting modes.

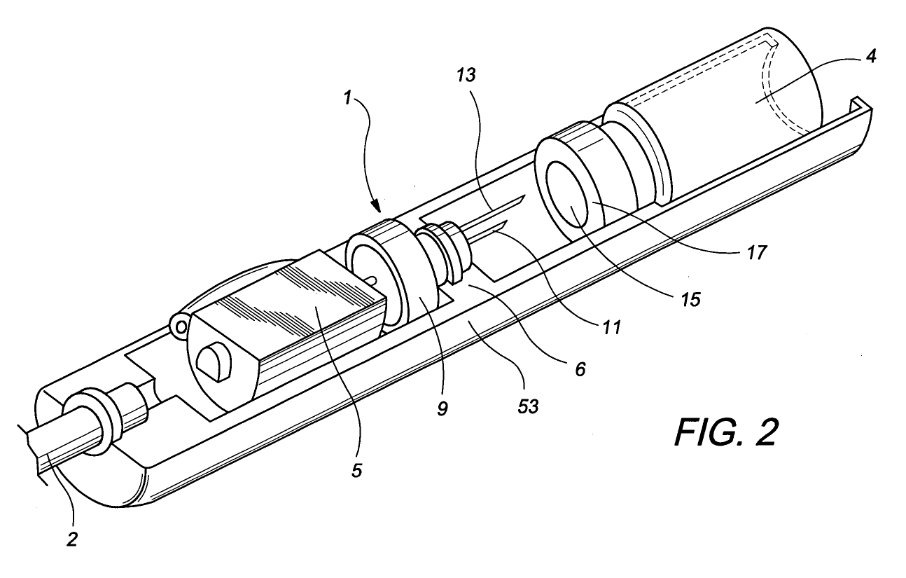

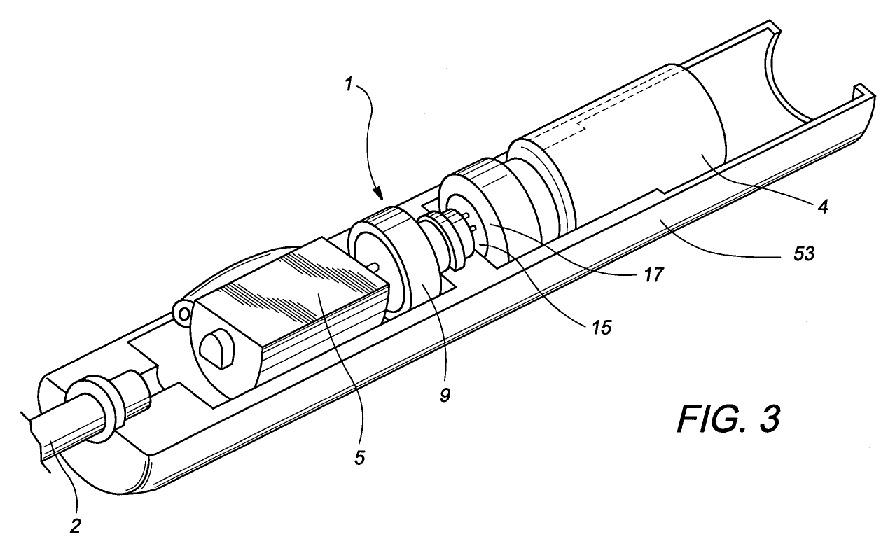

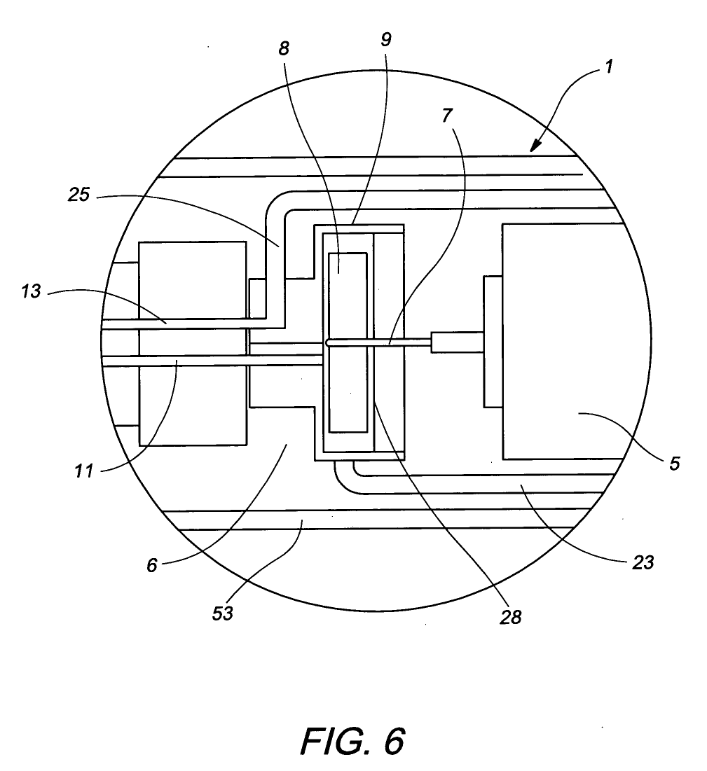

The instrument 12 comprises a handpiece 1, a shaft 2 and an electrode assembly 3 mounted at the distal end of the shaft. Referring to FIG. 2, the handpiece comprises a hollow housing 53, in which is located a fluid reservoir 4, a motor 5, and a connection block 6. Referring also to FIG. 6, the motor 5 includes a spindle 7, and a paddle wheel 8 attached to the spindle and located in a chamber 9 within the connection block 6. The connection block 6 also includes an inflow needle 11 and an outflow needle 13. The fluid reservoir 4 is slidable within the housing 53, between the position shown in FIG. 2 and that of FIG. 3, in which the inflow and outflow needles 11 and 13 pierce a diaphragm 15 present on the end face 17 of the fluid reservoir.

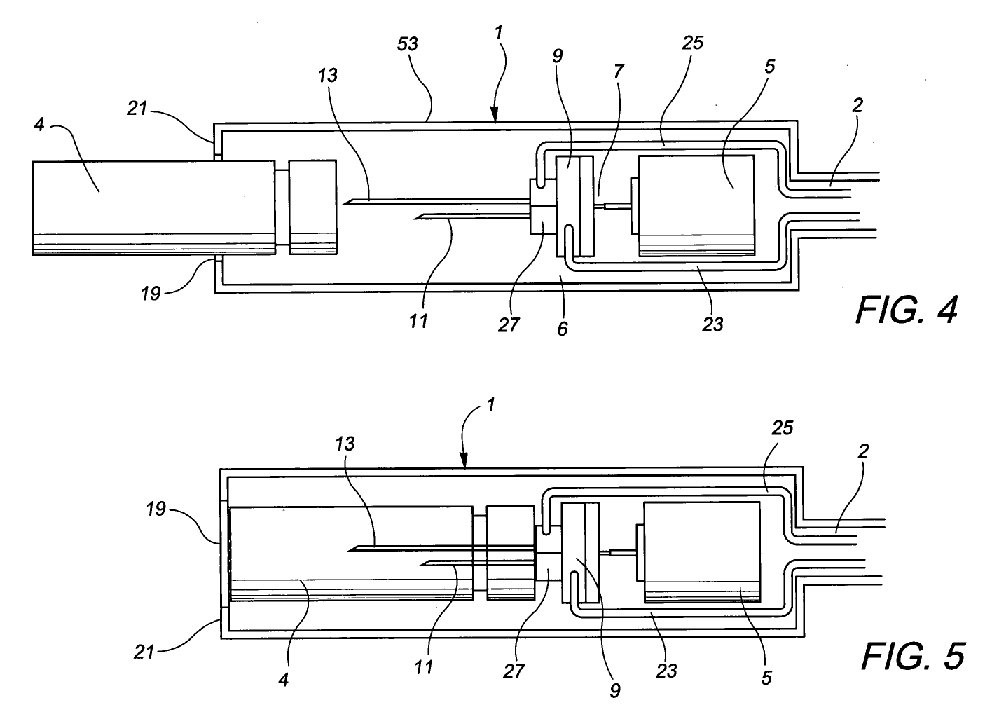

FIGS. 4 and 5 show an alternative version of the handpiece 1. In the handpiece 1 of FIGS. 4 and 5, the fluid reservoir 4 is introduced through an aperture 19 in the rear face 21 of the housing 53. FIGS. 4 and 5 also show a fluid feed line 23 and a fluid return line 25, which were omitted from FIGS. 2 and 3 for reasons of clarity. The fluid feed line 23 runs from the chamber 9, through the shaft 2, to the electrode assembly 3. The inflow needle 11 is in communication with the chamber 9, while the outflow needle 13 is in communication with fluid return line 25 at a section 27 of the connection block 6. The fluid return line 25 runs from the connection block 6, through the shaft 2, to the electrode assembly 3.

Referring to FIG. 6, the paddle wheel 8 is located in the chamber 9, and is mounted on the spindle 7, which spindle extends through a sealing membrane 28. The membrane 28 prevents cooling fluid from the chamber 9 entering the motor 5.

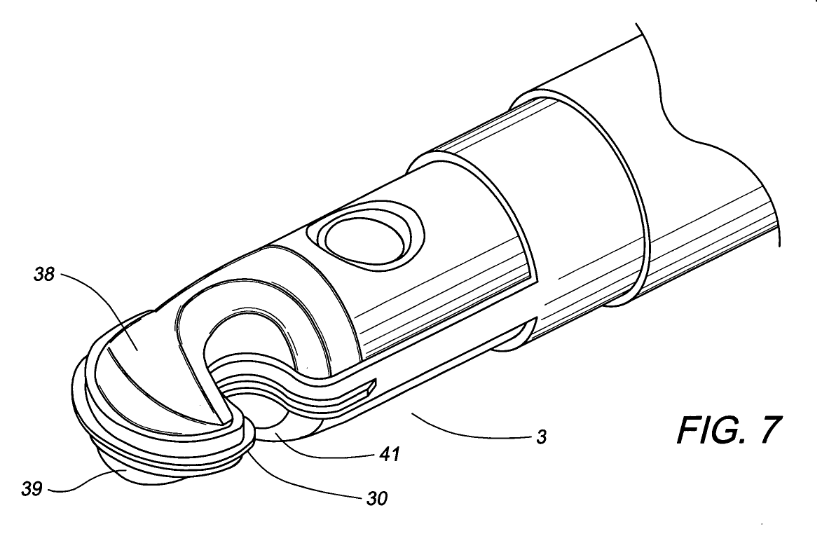

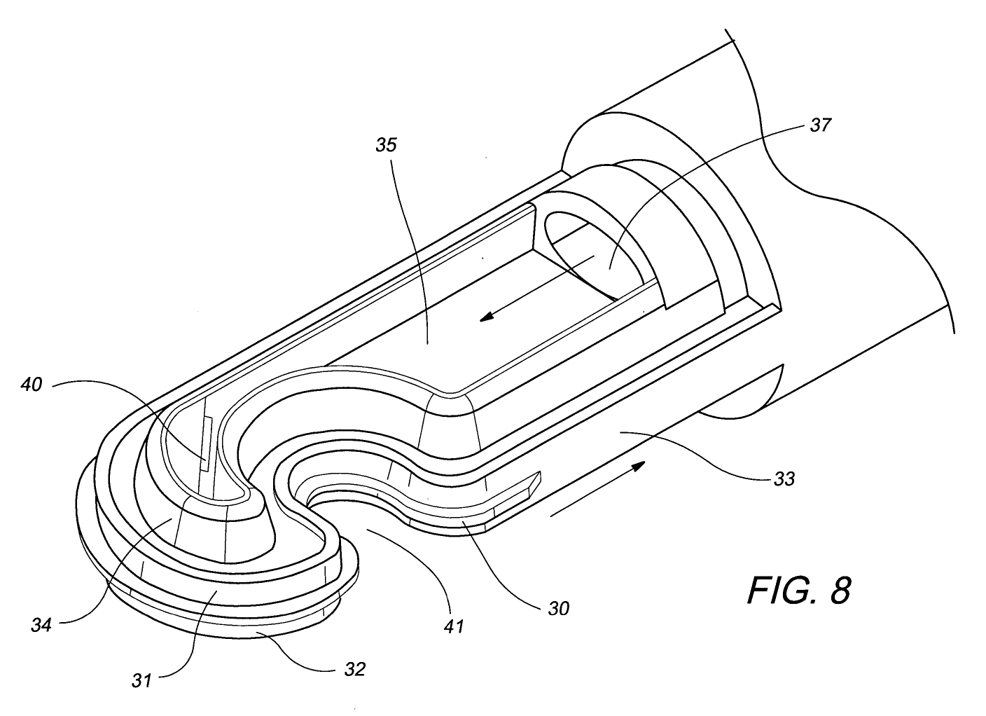

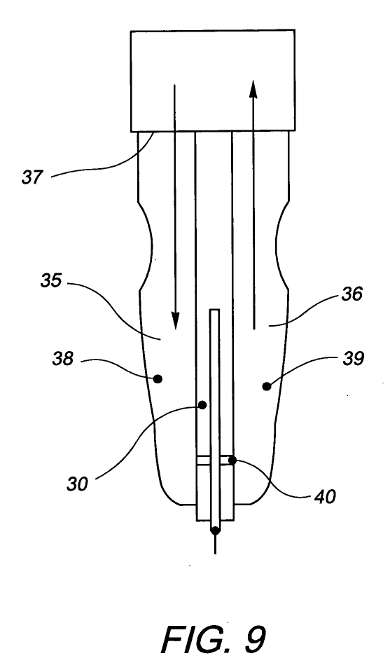

The electrode assembly 3 will now be described with reference to FIGS. 7 to 9. At the centre of the electrode assembly is a flat active electrode 30, with insulating mouldings 31 and 32 on either side thereof. The insulating mouldings 31 and 32 are both part of an integrated moulding assembly 33. The insulating moulding 31 includes wall portions 34 defining a hollow space 35 therein, while the insulating moulding 32 has similar wall portions defining a hollow space 36. The moulding 31 is provided with an opening 37 connecting the hollow space 35 with the fluid feed line 23, while the moulding 32 is provided with a similar opening connecting the hollow space 36 with the fluid return line 25.

The mouldings 31 and 32 are covered by electrically-conductive shells 38 and 39, constituting return electrodes for the electrode assembly 3. The active electrode 30 is provided with a through hole 40, connecting the hollow spaces 35 and 36 beneath the return electrodes 38 and 39. The electrode assembly 3 is in the form of a hook arrangement, with a recess 41 provided in one side thereof.

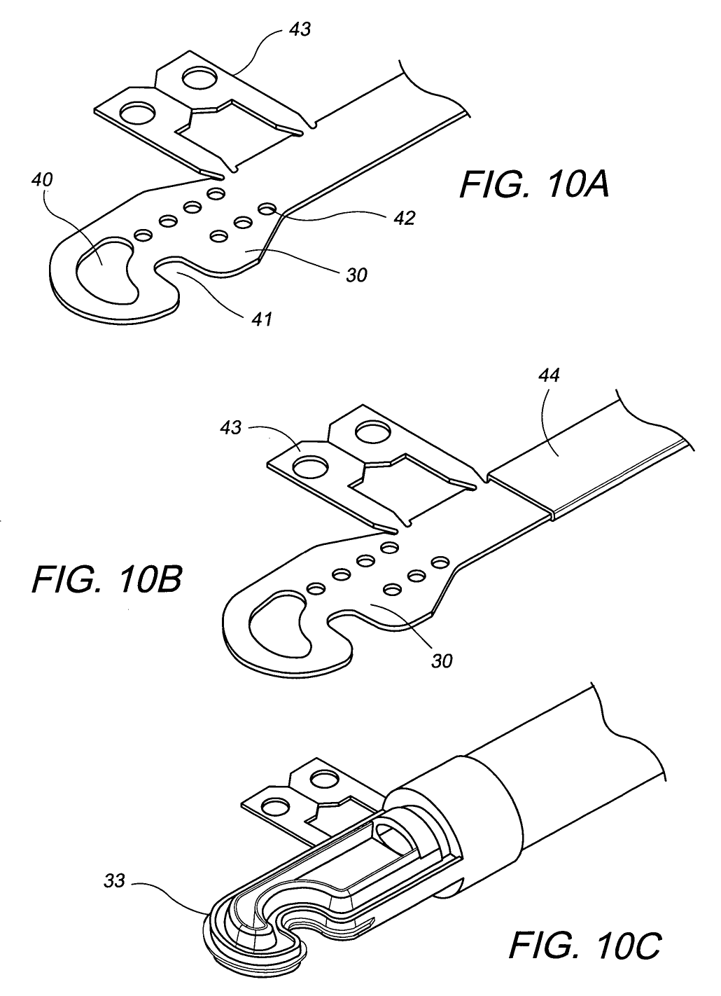

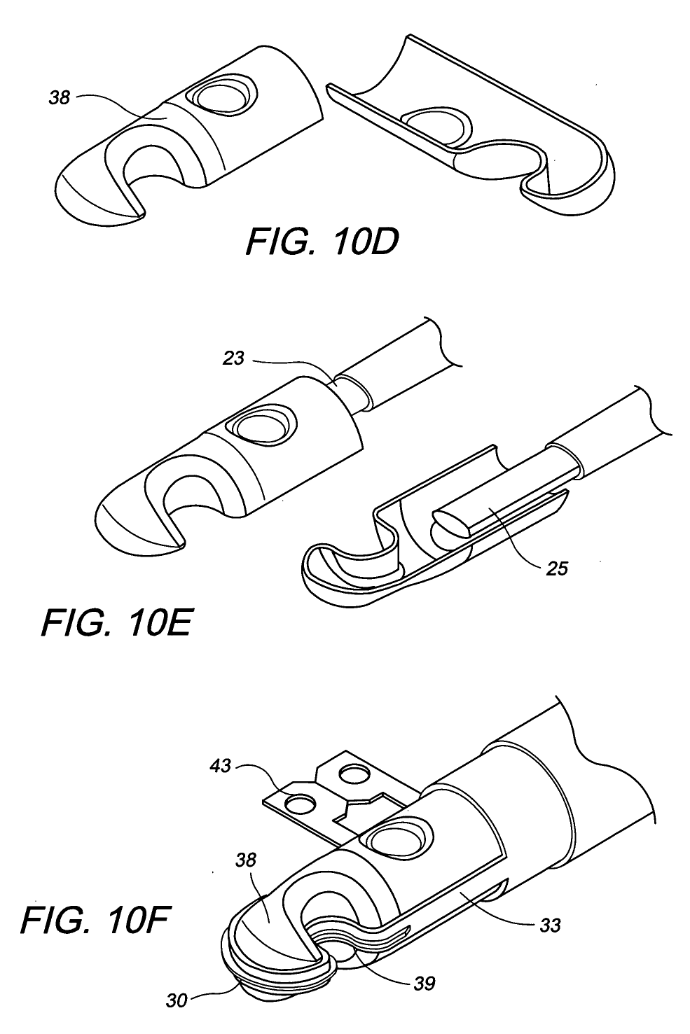

The assembly of the above construction will now be described with reference to FIGS. 10A to 10F. FIG. 10A shows the active electrode 30, formed by stamping from stainless steel. The stamped active electrode 30 has the through hole 40 formed therein, along with additional holes 42 provided for fastening purposes. The stamping also has ears 43, which are removed at the end of the manufacturing process, but which are provided for materials handling purposes.

FIG. 10B shows heat-shrink material 44 added to the proximal portion of the active electrode 30. The active electrode 30 is then assembled into the integrated moulding assembly 33, as shown in FIG. 10C. The insulating moulding assembly 33 is formed of ceramic, or alternatively silicone rubber. The electrically-conductive shells 38 and 39 are formed of copper (see FIG. 10D), and are assembled on to the moulding assembly 33 by welding them on to the metallic fluid feed and return lines 23 and 25 respectively (see FIG. 10E). The completed assembly is shown in FIG. 10F, prior to the removal of the ears 43.

The operation of the instrument 12 is as follows. If not already in position, the fluid reservoir 4 is moved into location with the connection block 6, as shown in FIGS. 3 and 5. The instrument 12 is connected to the generator 10, and introduced into the surgical site. The footswitch 16 is operated in order to supply an electrosurgical RF voltage to the electrodes 30, 38 and 39 in order to cut or coagulate tissue at the surgical site. The operation of the electrodes 30, 38 and 39 is described in more detail in our published application EP 1458300, but in essence when electrosurgical cutting is required a cutting voltage is supplied between the cutting electrode 30 and one or both of the return electrodes 38 and 39. Alternatively, when electrosurgical coagulation is required, a coagulating voltage is supplied between the return electrodes 38 and 39. In a blended mode, a blended waveform typically consisting of a waveform rapidly alternating between the cutting and coagulating voltage is supplied, typically also rapidly alternating between the cutting and coagulating electrode. For clarity, the leads connecting the RF signal between the cord 14 and the electrode assembly 3 have been omitted, but the fluid feed and return lines 23 and 25 could be formed of an electrically-conductive material and used for this purpose.

When the footswitch 16 is depressed, a signal is also sent to the motor 5 which causes the spindle 7 and hence the paddle wheel 8 to rotate. The rotation of the paddle wheel 8 causes cooling fluid to be driven out of the chamber 9 and through the fluid feed line 23. The cooling fluid is typically an electrically non-conductive fluid such as deionised water or ethanol. The cooling fluid travels though the fluid feed line 23 along the shaft 2 to the hollow space 35 within the return electrode 38. Once within the hollow space 35, the cooling fluid travels through the active electrode 30 by means of the through hole 40, and into the hollow space 36 within the other return electrode 39. From the hollow space 36, the cooling fluid travels back along the shaft 2 by means of the fluid return line 25 and into the reservoir 4 via the outflow needle 11.

The circulating cooling fluid travels to, and from, the electrode assembly 3, coming into close contact with both the return electrodes 38 and 39 and cooling them accordingly. By cooling the return electrodes 38 and 39, more electrical energy can be transferred into the tissue for coagulation purposes without the electrodes reaching a temperature at which tissue and blood will start to adhere to the electrode surfaces. It is essential that the cooling fluid is substantially electrically non-conductive, as it may come into contact with the active electrode 30 and with the return electrodes 38 and 39.

The motor 5 can be run continuously, or can be switched in and out whenever the electrode assembly 3 is actuated. In may be advantageous to run the motor 5, and hence circulate the cooling fluid, whenever the electrode assembly 3 is actuated, and for a predetermined additional time thereafter. In this way, any residual heat within the electrodes 30, 38 and 39, or transferred to the electrodes from adjacent hot tissue, will be removed by the cooling fluid.

It will be appreciated that the instrument 12 provides a handpiece 1 containing the fluid reservoir 4 and all of the fluid lines, and the only external lead is the connection cord 14 for the RF signal. This connection cord 14 can also be used for the electric supply to the motor 5. Alternatively, the RF signal can also be used as a supply for the motor 5. Heat is removed from the electrode assembly 3 by the cooling fluid, which is deposited back into the reservoir 4, and dissipated through the housing 53. For all normal operations of the instrument 12, the temperature rise of the housing 53 is only a few degrees, and still comfortable for the user of the instrument to hold.

By cooling the electrodes 30, 38 and 39, particularly during the coagulation of tissue, greater coagulative power can be applied without the overheating of the electrodes. Tissue sticking and the coating of the electrodes 30, 38 and 39 with dried blood are factors limiting the coagulative power of un-cooled instruments, and the present invention provides a compact and versatile instrument with considerable coagulative capabilities. In addition, the instrument, possibly even including the connection cord 14, can be made disposable, by the use of relatively-inexpensive components therein.

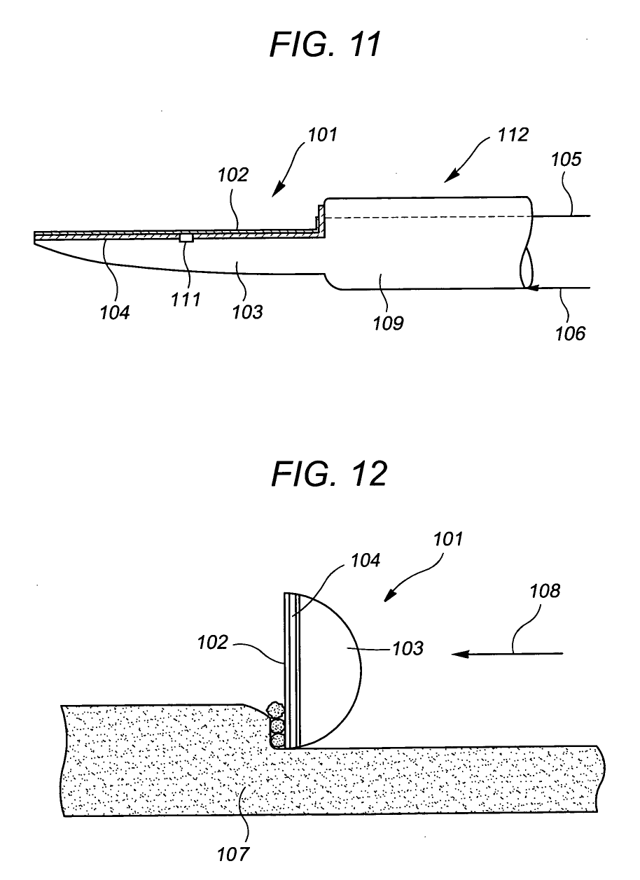

Referring to FIG. 11, the instrument 112 comprises a blade shown generally at 101 and including a generally flat first electrode 102, a larger second electrode 103, and an electrical insulator 104 separating the first and second electrodes. The first electrode 102 is formed of stainless steel having a thermal conductivity of 18 W/m.K (although alternative materials such as Nichrome alloy may also be used). The second electrode 103 is formed from a highly thermally-conducting material such as copper having a thermal conductivity of 400 W/m.K (alternative materials including silver or aluminium). The surface of the second electrode 103 is plated with a biocompatible material such as a chromium alloy, or with an alternative non-oxidising material such as nickel, gold, platinum, palladium, stainless steel, titanium nitride or tungsten disulphide. The electrical insulator 104 is formed from a ceramic material such as Al₂0₃ which typically has a thermal conductivity of 30 W/m.K. Other possible materials for the insulator 104 are available which have a substantially lower thermal conductivity. These include boron nitride, porcelain, steatite, Zirconia, PTFE, reinforced mica, silicon rubber or other ceramic materials such as foamed ceramics or mouldable glass ceramic such as that sold under the trademark MACOR.

A conductive lead 105 is connected to the first electrode 102, and a lead 106 is connected to the second electrode 103. The RF output from the generator 110 is connected to the blade 101 via the leads 105 and 106 so that a radio frequency signal having a substantially constant peak voltage (typically around 400V) appears between the first and second electrodes 102 and 103. Referring to FIG. 12, when the blade 101 is brought into contact with tissue 107 at a target site, the RF voltage will cause arcing between one of the electrodes and the tissue surface. Because the first electrode 102 is smaller in cross-sectional area, and has a lower thermal capacity and conductivity than that of the second electrode 103, the first electrode will assume the role of the active electrode and arcing will occur from this electrode to the tissue 107. Electrical current will flow through the tissue 107 to the second electrode 103, which will assume the role of the return electrode. Cutting of the tissue will occur at the active electrode, and the blade may be moved through the tissue. The blade 101 may be used to make an incision in the tissue 107, or moved laterally in the direction of the arrow 108 in FIG. 12 to remove a layer of tissue.

During cutting, considerable heat will be generated at the active electrode 102, and the electrode temperature may rise to 100-250° C. However, due to the poor thermal conductivity of the insulator 104, less heat is transmitted to the second electrode 103. Even when heat does reach the second electrode 103, the high thermal conductivity of the copper material means that much of the heat is conducted away from the electrode surface and into the body 109 of the electrode. This helps to ensure that a temperature differential is maintained between the first electrode 102 and the second electrode 103, and that the temperature of the second electrode 103 remains below 70° C. for as long as possible. This ensures that the second electrode 103 remains the return electrode whenever the instrument 12 is activated, and also that tissue does not begin to stick to the electrode 103.

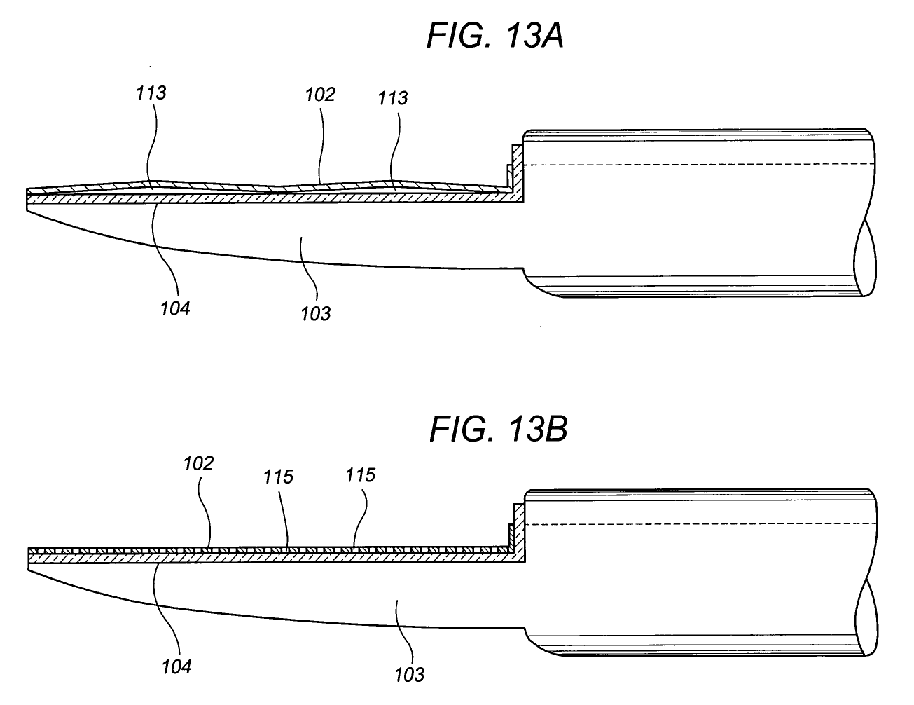

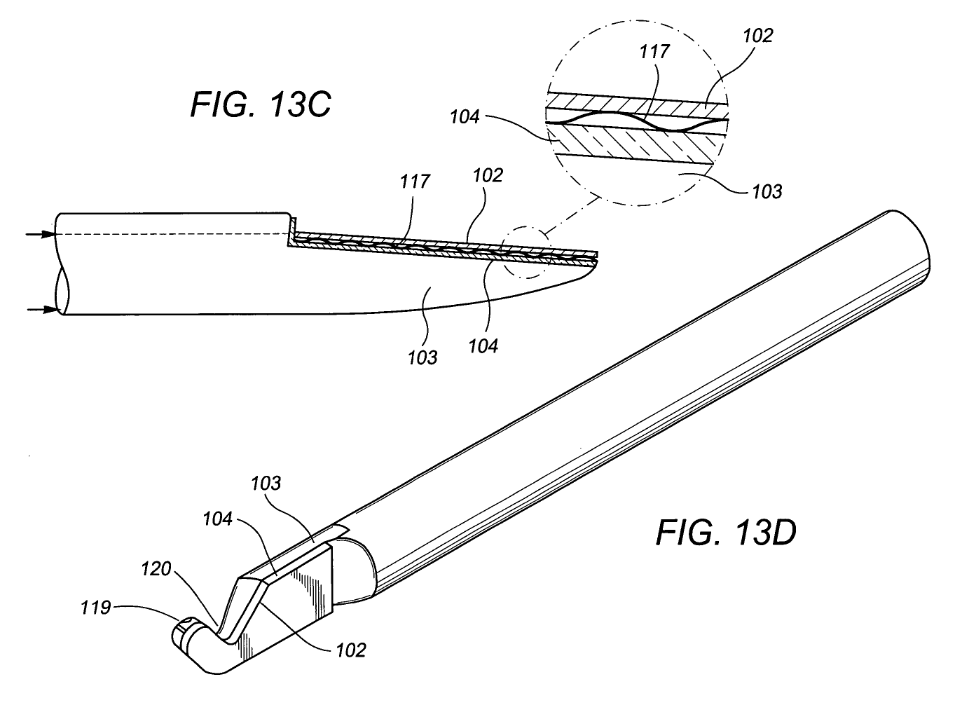

In addition to providing an insulator 104 which has a relatively low thermal conductivity, it is advantageous to ensure that the first electrode 102 contacts the insulator 104 as little as possible. In FIG. 11 the electrode 102 is not secured to the insulator 104 and the electrode 103 in a continuous fashion, but by one or more point contact pins shown generally at 111. FIG. 13a shows a further design of blade in which the first electrode 102 is shaped so as to contact the insulator 104 only intermittently along its length, with regions 113 over which the electrode bows outwardly from the insulator 104. This helps to minimise further the transfer of heat from the first electrode 102, through the insulator 104, to the second electrode 103. FIG. 13b shows a further arrangement in which the first electrode 102 is provided with many perforations 115 such that it is in the form of a mesh. Once again, this helps to minimise the transfer of heat from the first electrode 102 to the insulator 104. FIG. 13c shows another arrangement in which there is an additional corrugated electrode layer 117 located between the first electrode 102 and the insulator 104. As before, this assists in helping to prevent heat generated at the first electrode 102 from reaching the second electrode 103, so as to maintain the thermal differential therebetween.

FIG. 13d shows a variation on the blade of FIG. 11, in which the blade is formed as a hook 119. The first electrode 102, the second electrode 103 and the insulator 104 are all hook-shaped, and the operation of the device is substantially as described with reference to FIG. 11. The hook electrode is particularly suited for parting tissue, whether used as a cold resection instrument without RF energisation, or as an RF cutting instrument. Tissue may be held in the angle 120 of the hook 119, while being manipulated or cut.

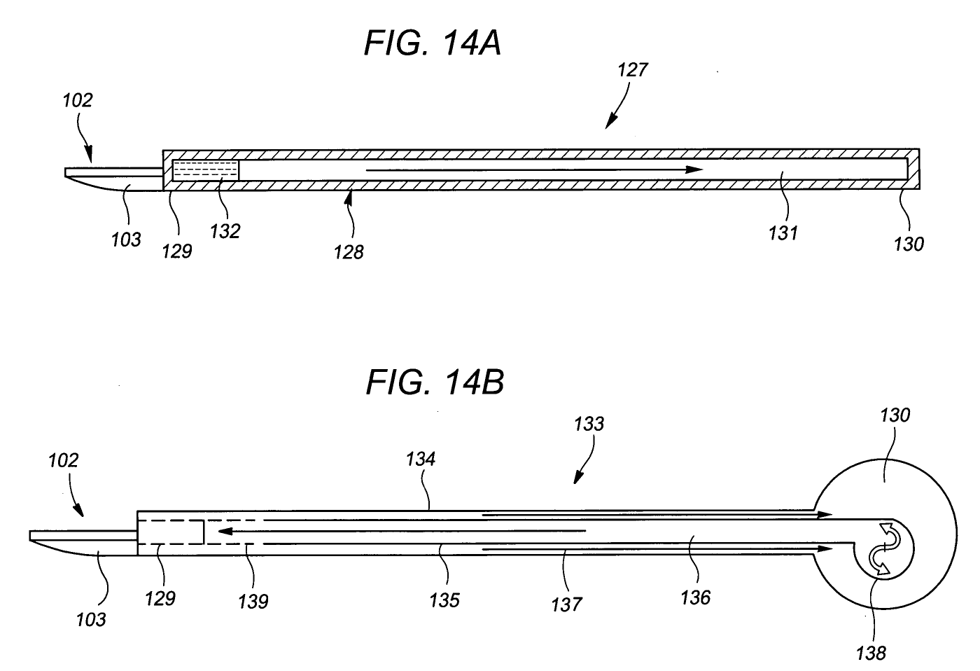

Whichever design of electrode is employed, it is advantageous if heat which does cross from the first electrode 102 to the second electrode 103 can be transferred away from the tissue contact surface of the electrode 103. In the blade of FIG. 11, the second electrode 103 is constituted by a relatively large mass of copper which is capable of conducting heat away from the electrode tip. The function of the electrode 103 can be further enhanced by employing cooling means as illustrated in FIGS. 14a and 14b. In FIG. 14a, the second electrode 103 is attached to a heat pipe shown generally at 127. The heat pipe 127 comprises a hollow closed tube 128 with a distal end 129 adjacent to the electrode 103, and a proximal end 130 within the handpiece of the instrument 112. The tube 128 has a cavity 131 therein, containing a low boiling temperature liquid 132 such as acetone or alcohol. In use, heat from the electrode 103 causes the liquid 132 at the distal end 129 of the tube to vaporise, and this vapour subsequently condenses at the proximal end 130 of the tube because it is relatively cool with respect to the distal end 129. In this way, heat is transferred from the distal end of the electrode 103 to the proximal end thereof, from where it can be further dissipated by the handpiece of the instrument 112.

FIG. 14b shows an alternative arrangement in which the heat pipe of FIG. 14a is replaced with a forced cooling system shown generally at 133. The cooling system 133 comprises a tube 134, again with a distal end 129 and a proximal end 130. The tube 134 includes a coaxial inner tube 135 defining an inner lumen 136 and an outer lumen 137. The inner tube 135 is perforated towards the distal end of the tube, so that the inner and outer lumens 136 and 137 are in communication one with another. In use, a self-contained pump 138 causes a cooling fluid 139 to be circulated up the inner lumen 136 to the distal end 129, returning via the outer lumen 137 to be recirculated continuously. The circulating fluid is heated by the electrode 103, and the heat is taken by the fluid to the proximal end 130 of the tube 134. In this way, the second electrode 103 is kept cool, despite the elevated temperature at the first electrode 102.

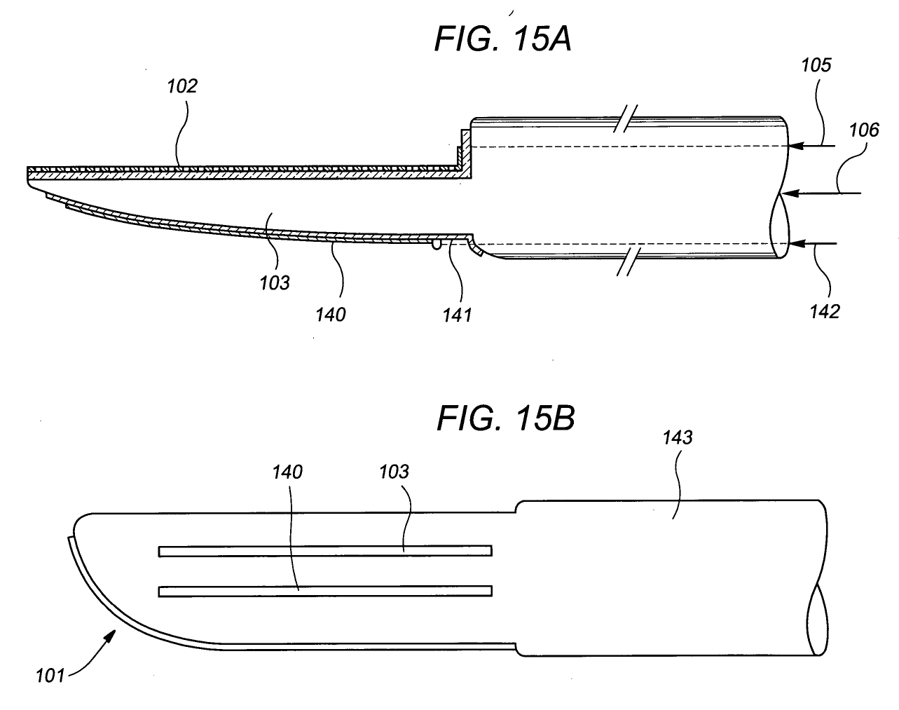

The remainder of the Figures show arrangements in which a third electrode 140 is provided, in order to allow the coagulation or desiccation of the tissue 107. In FIG. 15a, a blade 101 is shown in accordance with the construction of FIG. 13b, and like parts are designated with like reference numerals. The third electrode 140 is attached to the second electrode 103, on the opposite side to the first electrode 102, and mounted on a further electrical insulator 141. RF signals may be supplied to the third electrode 140 from the generator 110 via a lead 142. The insulator 141 is formed from a thin layer of silicon rubber, alternative materials for the insulator 141 including polyamide, PEEK or PVC materials. The thin layer ensures that heat can transfer across the silicon rubber layer and that the coagulation electrode 140 can benefit from the thermal conductivity properties of the second electrode 103. In this way, the coagulation electrode 140 can remain relatively cool despite any heat previously generated by the first electrode 102. In use, tissue is cut as previously described. When it is desired to coagulate instead of cutting, the third electrode 140 is placed in contact with the tissue 107, and a coagulating RF signal is applied between the second electrode 3 and the third electrode 140.

FIG. 15b shows an alternative embodiment in which the second electrode 103 and third electrode 140 are metallised tracks on a substrate 143 of aluminium nitride material. As before, this material is electrically insulating yet a good thermal conductor, to allow for the conduction of heat away from the second and third electrodes.

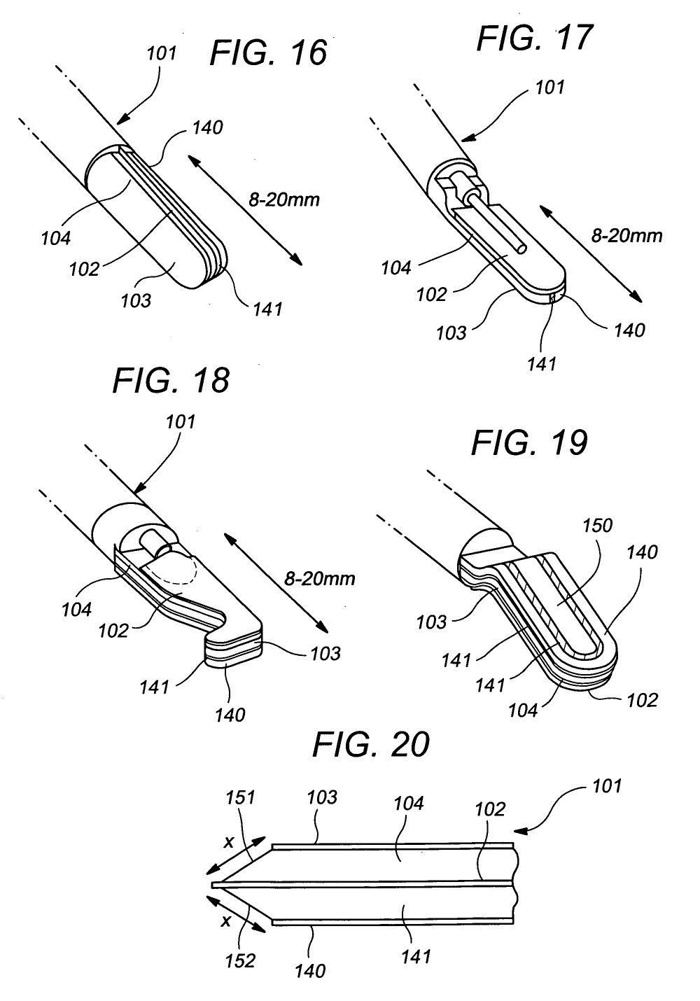

FIG. 16 shows an arrangement in which the first electrode 102 is located between the second and third electrodes 103 and 140. Both the electrodes 103 and 140 are approximately semi-circular in cross-section, and form a generally cylindrical structure with the first electrode 102 protruding slightly from the central region thereof. The insulating layer 104 separates the first electrode 102 from the second electrode 103, and the insulating layer 141 separates the first electrode 102 from the third electrode 140. When the user intends the instrument to cut tissue, the generator 110 applies a cutting RF signal between the first electrode 102 and one or both of the second or third electrodes 103, 140. Conversely, when the user intends the instrument to coagulate tissue, the generator 110 applies a coagulating RF signal between the second electrode 103 and the third electrode 140. The relatively large surface area of the electrodes 103 and 140 allows for effective coagulation of tissue, as well as for the conduction away of heat during cutting as previously described.

FIG. 17 shows an alternative design of instrument in which the second and third electrodes 103 and 140 are provided side-by-side. The first electrode 102 is substantially planar, and an insulating layer 104 separates the first electrode from the second and third electrodes 103 and 140 on the other side of the instrument. The electrodes 103 and 140 are disposed in side-by-side arrangement, with an insulating section 141 therebetween. As before, the instrument can cut tissue with an RF signal between the first electrode 102 and one of the second or third electrodes 103, 140, or alternatively coagulate tissue with an RF signal between the second and third electrodes.

FIG. 18 shows a further embodiment in which the first, second and third electrodes are provided as a series of layers in a “sandwich” arrangement. The first electrode 102 is shown as the top layer in FIG. 18, with the third electrode 140 as the bottom layer, with the second electrode 103 sandwiched therebetween. Insulating layers 104 and 141 respectively serve to separate the first, second, and third electrodes. This arrangement provides a relatively thick edge to the blade 1, which is designed to facilitate coagulation of tissue.

FIG. 19 shows an arrangement which utilises features from both the sandwich and side-by-side electrode structures. The electrodes are again provided in a sandwich arrangement, FIG. 19 showing the first electrode 102 on the bottom rather than the top as shown in FIG. 109. The second electrode 103 is again in the middle of the sandwich, separated from the first electrode by an insulating layer 104. The third electrode 140 is shown as the top electrode in FIG. 19, but has a central recess though which a raised portion 150 of the second electrode 103 can protrude. The second and third electrodes are separated by an insulator 141, and the top surface of the protrusion 150 is flush with the top of the third electrode 140. This arrangement allows either the sides of the blade 101 or the top face as shown in FIG. 110 to be used for the coagulation of tissue.

FIG. 20 shows an arrangement in which the end of the blade 101 comprises a central first electrode 102 with insulating layers 104 and 141 on either side thereof. The insulating layers 104 and 141 each have a slanting beveled distal end, as shown at 151 and 152 respectively. A second electrode 103 is attached to the insulating layer 104, the beveled end 151 resulting in the second electrode being set back axially from the first electrode 102 in the axis of the blade. In similar fashion, a third electrode 140 is attached to the insulating layer 141, the beveled end 152 resulting in the third electrode also being axially set back from the first electrode 102. The beveled ends 151 and 152 allow for a minimum separation (shown at “x” in FIG. 20) of 0.25 mm between the first electrode and the second and third electrodes, while maintaining an overall slim profile to the blade 101. The first electrode 102 can be flush with the ends of the first and second insulating layers 104 and 141, or may project slightly there from as shown in FIG. 20. As described previously, the transfer of heat by the first electrode can be reduced by a number of techniques, including attaching it to the insulating layers in a discontinuous manner, or perforating it with a plurality of holes in order to reduce heat transfer.

The invention relies on the careful selection of a number of design parameters, including the spacing between the first and second electrodes, the voltage supplied thereto, the size and materials selected for the electrodes, and for the electrical insulator or insulators. This careful selection should ensure that there is no direct arcing between the electrodes, that only one electrode is encouraged to be the active electrode, and that the return electrode is kept cool either by preventing heat reaching it and/or by transferring heat away from it should the heat reach the second electrode.

The relatively cool return electrode ensures that there is relatively little or no thermal damage to tissue adjacent the return of the instrument, while the tissue assists in the conduction of heat away from the return.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

What is claimed is:

1. An electrosurgical instrument comprising a handpiece, an electrode assembly comprising one or more electrodes attached to the handpiece, and connection means for connecting the handpiece to an electrosurgical generator, the handpiece comprising a housing, fluid supply lines for directing a cooling fluid to and from the electrode assembly, and a pump for driving cooling fluid through the fluid supply lines, the pump and the fluid supply lines both being wholly contained within the housing.

2. An electrosurgical instrument according to claim 1, wherein the housing contains a reservoir of cooling fluid.

3. An electrosurgical instrument according to claim 2, wherein the handpiece is such that there are two possible arrangements for the fluid reservoir, a first arrangement in which the reservoir is not connected to the fluid supply lines, and a second arrangement in which the reservoir is connected to the fluid supply lines.

4. An electrosurgical instrument according to claim 3, wherein the housing is such that the reservoir is movable between first and second positions, the first position being in which the reservoir is not connected to the fluid supply lines, and the second position being in which the reservoir is connected to the fluid supply lines.

5. An electrosurgical instrument according to claim 1, wherein the electrode assembly comprises at least two electrodes separated by an insulating spacer.

6. An electrosurgical instrument according to claim 5, wherein the electrode assembly comprises three electrodes provided in a sandwich structure with insulating layers therebetween.

7. An electrosurgical instrument according to claim 1, wherein the electrode assembly is in the form of a substantially flat blade.

8. An electrosurgical instrument according to claim 1, wherein the pump is driven by an electric motor.

9. An electrosurgical instrument according to claim 8, wherein the electric motor is a synchronous motor.

10. An electrosurgical instrument according to claim 8, wherein the electric motor constitutes the pump.

11. An electrosurgical instrument according to claim 10, wherein the motor includes a spindle on which is provided a paddle, the paddle being rotatable by the motor.

12. An electrosurgical system comprising an electrode assembly comprising one or more electrodes, a handpiece to which the electrode assembly is secured, an electrosurgical generator for supplying a radio frequency voltage signal to the electrode assembly, and a cooling system for cooling the electrode assembly, the cooling system including fluid supply lines and a pump for driving cooling fluid through the fluid supply lines, the cooling system being wholly contained within the handpiece and the electrode assembly.

13. An electrosurgical handpiece comprising a housing, first connection means for attaching an electrode assembly to the handpiece, second connection means for connecting the handpiece to an electrosurgical generator, fluid supply lines for directing a cooling fluid to and from the electrode assembly, and a pump for driving cooling fluid through the fluid supply lines, the pump and the fluid supply lines both being wholly contained within the housing of the handpiece.
